(19) United States
(12) Patent Application Publication
(10) Pub. No.: US 2022/0312932 A1
(43) Pub. Date: Oct. 6, 2022

(54) HAIR-INCREASING FIBER POWDER PACKAGING BOTTLE WITH MANUALLY ADJUSTABLE FIBER AMOUNT

(71) Applicant: Zhanchun Zhao, Wuchuan City (CN)

(72) Inventor: Zhanchun Zhao, Wuchuan City (CN)

(21) Appl. No.: 17/216,720

(22) Filed: Mar. 30, 2021

(51) Int. Cl. A45D 33/02; B65D 47/12; B65D 47/26; G01F 13/00; A61K 8/02; A61Q 5/06

(52) U.S. Cl. CPC A45D 33/02; B65D 47/122; B65D 47/261; G01F 13/001; A61K 8/027; A61Q 5/06

(57) ABSTRACT

A hair-increasing fiber powder packaging bottle with a manually adjustable fiber amount includes a packaging bottle body. The packaging bottle body includes a bottle cap, a top screw cap, a lower cap body, a filter plug, and a bottle body. The top screw cap is mounted to a bottom end of the bottle cap. A deformation support and a non-slip surface are disposed on two sides of the top screw cap. A deformation space is disposed on an inner side of the deformation support. A surface of the top screw cap is provided with a discharge hole. A gear projection is mounted to one side of the top screw cap. A connecting projection is disposed on a side edge of the top screw cap. A sealing boss is disposed on a top end of the top screw cap.

### TECHNICAL FIELD

The present disclosure relates to the field of processing and packaging technologies, and in particular, to a hair-increasing fiber powder packaging bottle with a manually adjustable fiber amount.

### BACKGROUND

Currently, the social pressure is growing. Due to some external reasons such as study or work pressure, hereditary hair loss and hormonal hair loss occur. As a result, the number of groups with thinning hair is increasing. Therefore, a hair-increasing fiber powder product is available on the market. As long as the product is scattered on a thinning hair area, there is a ready-made thick hair visual effect. However, during use, a fiber amount needed is different for each group with thinning hair. Groups with relatively thinning hair need to use more fiber, while those with not very thinning hair need to use less fiber. At present, there is no device with manually adjustable fiber output on the market, which cannot solve the problem of different amount among different groups.

### SUMMARY

With respect to the shortcomings in the prior art, an objective of the present disclosure is to provide a hair-increasing fiber powder packaging bottle with a manually adjustable fiber amount, so as to solve the problem raised in said BACKGROUND. According to the present disclosure, output of hair-increasing fiber powder can be manually controlled, the fiber can be more uniformly spread on the head, the effect is more realistic, the consumption is more economical, and the use efficiency of the fiber is improved.

In order to achieve the above objective, the present disclosure is achieved through the following technical solution: a hair-increasing fiber powder packaging bottle with a manually adjustable fiber amount, including a packaging bottle body. The packaging bottle body includes a bottle cap, a top screw cap, a lower cap body, a filter plug, and a bottle body. The top screw cap is mounted to a bottom end of the bottle cap. A deformation support and a non-slip surface are disposed on two sides of the top screw cap. A deformation space is disposed on an inner side of the deformation support. A discharge hole is defined on a surface of the top screw cap. A gear projection is mounted to one side of the top screw cap. A connecting projection is disposed on a side edge of the top screw cap. A sealing boss is disposed on a top end of the top screw cap. The lower cap body is mounted to a bottom end of the top screw cap. A sealing groove is defined on a top end of the lower cap body. A connecting groove and a gear groove are defined on a side edge of the lower cap body. A reinforcing rib limit is disposed on two ends of the gear groove. The filter plug is disposed at a bottom end of the lower cap body. The bottle body is disposed at a bottom end of the filter plug.

As a preferred implementation of the present disclosure, five gear grooves are provided.

As a preferred implementation of the present disclosure, the sealing boss is matched and sealed with the sealing groove.

As a preferred implementation of the present disclosure, the connecting projection is stuck inside the connecting groove.

As a preferred implementation of the present disclosure, the lower cap body and the filter plug are installed and fixed by interference fit.

As a preferred implementation of the present disclosure, a surface of the filter plug is provided with a plurality of holes.

The present disclosure has the following beneficial effects: a hair-increasing fiber powder packaging bottle with a manually adjustable fiber amount according to the present disclosure includes a packaging bottle body. The packaging bottle body includes a bottle cap, a top screw cap, a lower cap body, a filter plug, a bottle body, a deformation space, a deformation support, a sealing boss, a connecting projection, a sealing groove, a connecting groove, a discharge hole, a non-slip surface, a gear projection, a gear groove, and a reinforcing rib limit.

1. According to the hair-increasing fiber powder packaging bottle with a manually adjustable fiber amount, a gear of fiber output suitable for a person can be selected according to a thinning degree of hair of the person. Adjustment of the gear is simple and convenient.

2. The hair-increasing fiber powder packaging bottle with a manually adjustable fiber amount enables the fiber powder to be more uniformly spread on the human head, and the effect is more realistic.

3. The hair-increasing fiber powder packaging bottle with a manually adjustable fiber amount can make the consumption more economical, and the use of the fiber is more efficient.

### BRIEF DESCRIPTION OF THE DRAWINGS

FIG. 1 is a schematic composite structure diagram of an appearance of a hair-increasing fiber powder packaging bottle with a manually adjustable fiber amount according to the present disclosure;

FIG. 2 is a schematic diagram of cooperation between a top screw cap and a lower cap body of the hair-increasing fiber powder packaging bottle with a manually adjustable fiber amount according to the present disclosure;

FIG. 3 is a cross-sectional schematic diagram taken along the line III-III shown in FIG. 2;

FIG. 4 is a schematic structural diagram of the top screw cap of the hair-increasing fiber powder packaging bottle with a manually adjustable fiber amount according to the present disclosure;

FIG. 5 is a cross-sectional schematic diagram taken along the line V-V shown in FIG. 4;

FIG. 6 is a structural schematic diagram of the lower cap body of the hair-increasing fiber powder packaging bottle with a manually adjustable fiber amount according to the present disclosure;

FIG. 7 is a taken along the line VII-VII shown in FIG. 6; and

FIG. 8 is a schematic structural diagram of a filter plug of the hair-increasing fiber powder packaging bottle with a manually adjustable fiber amount according to the present disclosure.

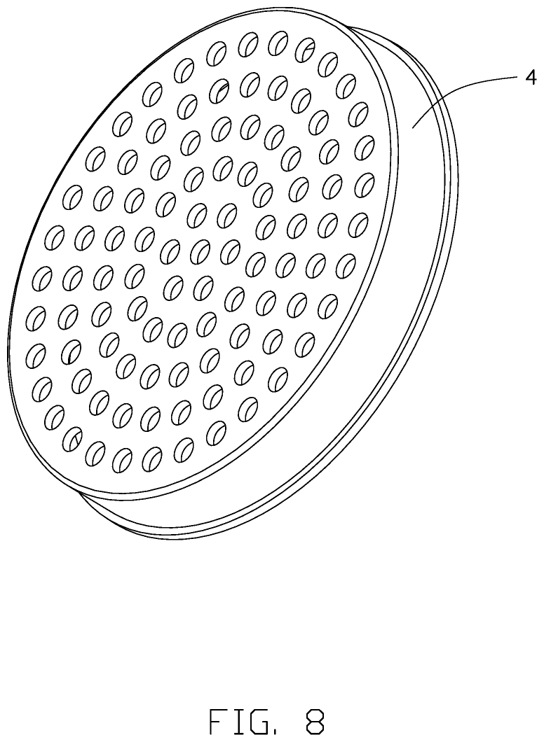

In the drawings, 1. bottle cap; 2. top screw cap; 3. lower cap body; 4. filter plug; 5. bottle body; 6. deformation space; 7. deformation support; 8. sealing boss; 9. connecting projection; 10. sealing groove; 11. connecting groove; 12. discharge hole; 13. non-slip surface; 14. gear projection; 15. gear groove; 16. reinforcing rib limit.

### DETAILED DESCRIPTION OF THE EMBODIMENTS

In order to make the technical means, creation features, achieved objectives, and effects of the present disclosure easy to understood, the present disclosure is further described below with reference to specific implementations.

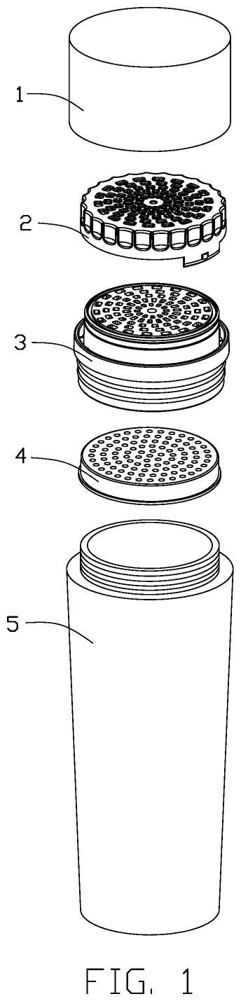

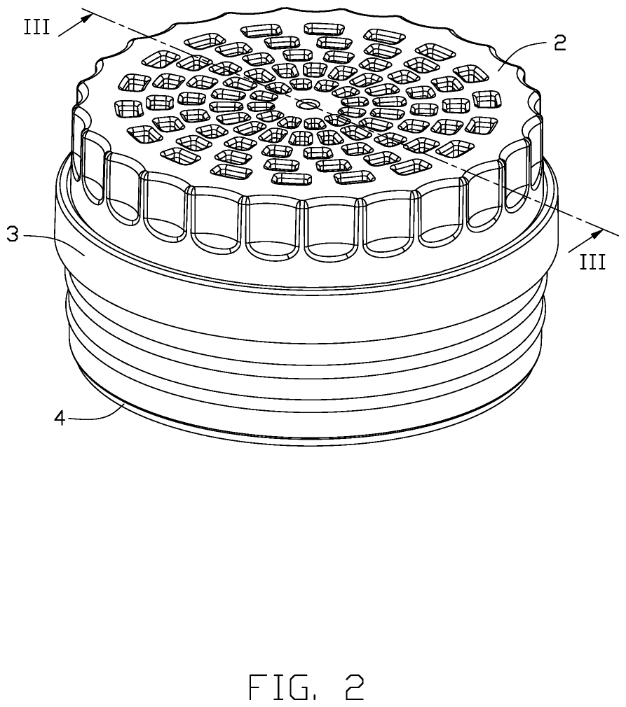

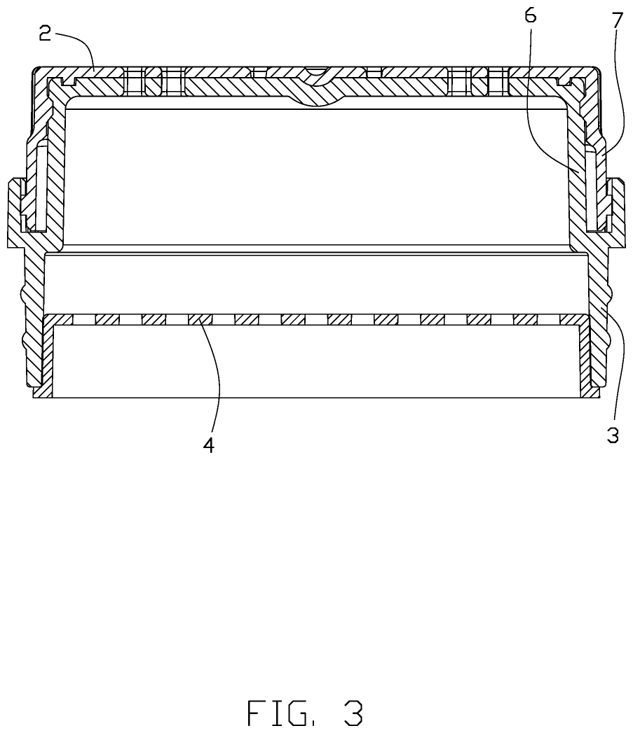

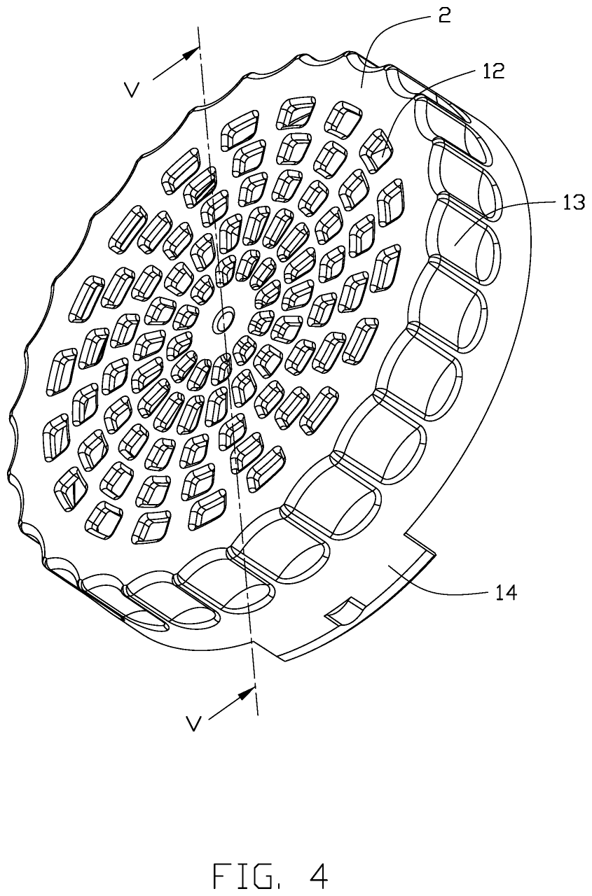

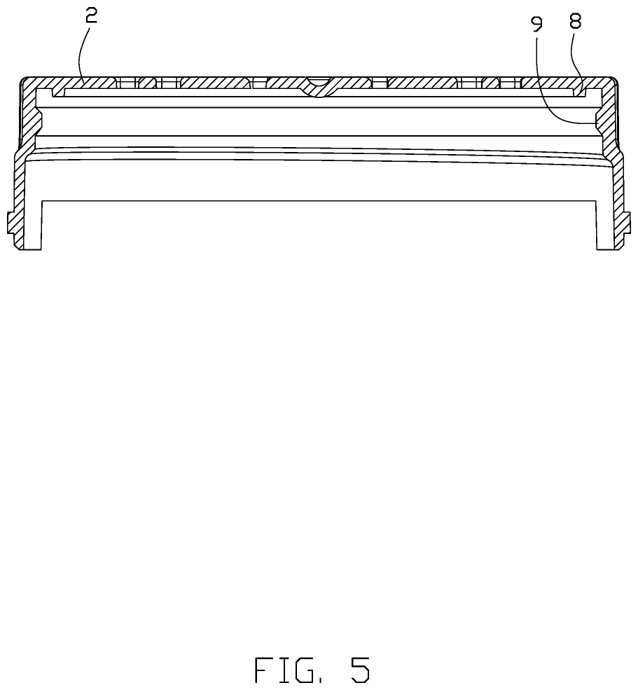

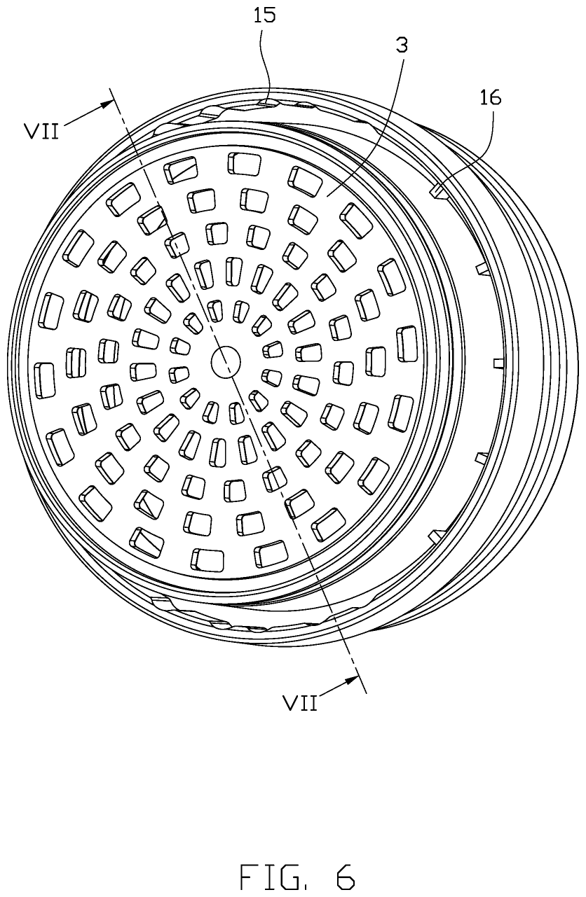

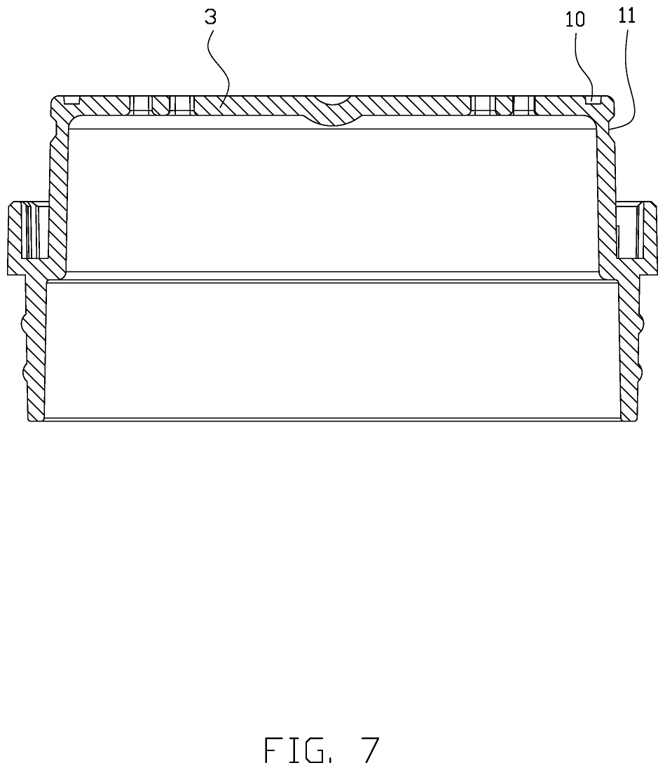

Referring to FIG. 1 to FIG. 7, the present disclosure provides a technical solution: a hair-increasing fiber powder packaging bottle with a manually adjustable fiber amount, including a packaging bottle body. The packaging bottle body includes a bottle cap 1, a top screw cap 2, a lower cap body 3, a filter plug 4, and a bottle body 5. The top screw cap 2 is mounted to a bottom end of the bottle cap 1, a deformation support 7 and a non-slip surface 13 are disposed on two sides of the top screw cap 2, a deformation space 6 is disposed on an inner side of the deformation support 7, a discharge hole 12 is defined on a surface of the top screw cap 2, a gear projection 14 is mounted to one side of the top screw cap 2, a connecting projection 9 is disposed on a side edge of the top screw cap 2, a sealing boss 8 is disposed on a top end of the top screw cap 2, the lower cap body 3 is mounted to a bottom end of the top screw cap 2, a sealing groove 10 is defined on a top end of the lower cap body 3, a connecting groove 11 and a gear groove 15 are defined on a side edge of the lower cap body 3, a reinforcing rib limit 16 is disposed on two ends of the gear groove 15, the filter plug 4 is disposed at a bottom end of the lower cap body 3, and the bottle body 5 is disposed at a bottom end of the filter plug 4.

As a preferred implementation of the present disclosure, five gear grooves 15 are provided. The gear groove 15 and the gear projection 14 deform when cooperating to rotate to increase a sense of gear.

As a preferred implementation of the present disclosure, the sealing boss 8 is matched and sealed with the sealing groove 10 to seal the fiber inside the bottle body 5 to prevent sprinkling.

As a preferred implementation of the present disclosure, the connecting projection 9 is stuck inside the connecting groove 11 to integrally connect the top screw cap 2 and the lower cap body 3.

As a preferred implementation of the present disclosure, the lower cap body 3 and the filter plug 4 are installed and fixed by interference tit, and a bottom end of the filter plug 4 is provided with two step limits to provide greater stability and convenience.

As a preferred implementation of the present disclosure, a surface of the filter plug 4 is provided with a plurality of holes to control the flow, and two spaces are formed by blocking the space to improve stability.

Working principle is as follows. The hair-increasing fiber powder packaging bottle with a manually adjustable fiber amount mainly consists of the bottle cap 1, the top screw cap 2, the lower cap body 3, the filter plug 4, and the bottle body 5. During assembly, the top screw cap 2 and the lower cap body 3 cooperate with and are connected to the connecting projection 9 and the connecting groove 11, and two centers of circle of the top screw cap 2 and the lower cap body 3 are offset but do not coincide to facilitate mounting and tensioning. The sealing boss 8 and the sealing groove 10 are disposed at a connecting portion between the top screw cap 2 and the lower cap body 3 to prevent the fiber powder from leaking between the two. The filter plug 4 and the lower cap body 3 are fastened by interference fit. After the assembly of the top screw cap 2 and the lower cap body 3, a size of the discharge hole 12 can be adjusted by rotating the top screw cap 2 to control the fiber powder output. When the discharge hole is fully opened, the maximum powder output is at the position of 0°, and there are other gears are at positions of 12°, 26°, 35°, and 50°. When the gear reaches the position of 50°, the discharge hole 12 is completely closed, and positions of 0-50° are controlled within a range of the gears by the reinforcing rib limit 16. The gear groove 15 and the gear projection 14 deform when cooperating to rotate to increase a sense of gear. The gear projection 14 independently protrudes to reduce an area to be easier to deform, with the deformation space 6 and the deformation support 7 reserved inside to facilitate better deformation. The top screw cap 2 is provided with a non-slip surface, so that the friction is increased during rotation. In use, the bottle cap 1 is opened, the top screw cap 2 is rotated, the top screw cap 2 and the lower cap body 3 may be staggered to change an area of the discharge hole 12, so that a suitable gear is found to shake the bottle up and down and spread the fiber powder evenly on human heads. The filter plug 4 plays a role of controlling the air pressure in the bottle body 5 to prevent the negative pressure in the bottle from impeding the discharge of the fiber. When shaking the bottle up and down, the air passes through the filter plug 4 into the bottle body 5. At the same time, the size of the hole in the filter plug 4 can be changed to control air flow. Further, two spaces are formed by blocking one space to improve stability, sudden spilling of the fiber is prevented during first use so that users may have better experience.

The above shows and describes the basic principles, main features and advantages of the present disclosure. It will be apparent to those skilled in the art that the present disclosure is not limited to the details of the above exemplary embodiments and that the present disclosure may be realized in other specific forms without departing from the spirit or essential characteristics of the present disclosure. Accordingly, the embodiments herein are to be considered as exemplary and not restrictive, and the scope of the present disclosure is defined by the appended claims rather than by the above description. It is therefore intended that all changes that come within the meaning and range of equivalents to the claims are intended to be embraced within the scope of the present disclosure. Any reference numerals in the claims should not be construed as limiting the involved claims.

In addition, it should be understood that the specification is described according to the implementations, but it is not necessary that each implementation includes only one independent technical solution. Such representation of the specification is for clarity only. Those skilled in the art shall take the specification as a whole, and the technical solutions in the embodiments may also be suitably combined to form other implementations that can be understood by those skilled in the art.

What is claimed is:

1. A hair-increasing fiber powder packaging bottle with a manually adjustable fiber amount, comprising a packaging bottle body, wherein the packaging bottle body comprises a bottle cap (1), a top screw cap (2), a lower cap body (3), a filter plug (4), and a bottle body (5), the top screw cap (2) is mounted to a bottom end of the bottle cap (1), a deformation support (7) and a non-slip surface (13) are disposed on two sides of the top screw cap (2), a deformation space (6) is disposed on an inner side of the deformation support (7), a discharge hole (12) is defined on a surface of the top screw cap (2), a gear projection (14) is mounted to one side of the top screw cap (2), a connecting projection (9) is disposed on a side edge of the top screw cap (2), a sealing boss (8) is disposed on a top end of the top screw cap (2, the lower cap body (3) is mounted to a bottom end of the top screw cap (2), a sealing groove (10) is defined on a top end of the lower cap body (3), a connecting groove (11) and a gear groove (15) are defined on a side edge of the lower cap body (3), a reinforcing rib limit (16) is disposed on two ends of the gear groove (15), the filter plug (4) is disposed at a bottom end of the lower cap body (3), and the bottle body (5) is disposed at a bottom end of the filter plug (4).

2. The hair-increasing fiber powder packaging bottle with a manually adjustable fiber amount according to claim 1, wherein five gear grooves (15) are provided.

3. The hair-increasing fiber powder packaging bottle with a manually adjustable fiber amount according to claim 1, wherein the sealing boss (8) is matched and sealed with the sealing groove (10).

4. The hair-increasing fiber powder packaging bottle with a manually adjustable fiber amount according to claim 1, wherein the connecting projection (9) is stuck inside the connecting groove (11).

5. The hair-increasing fiber powder packaging bottle with a manually adjustable fiber amount according to claim 1, wherein the lower cap body (3) and the filter plug (4) are installed and fixed by interference fit.

6. The hair-increasing fiber powder packaging bottle with a manually adjustable fiber amount according to claim 1, wherein a surface of the filter plug (4) is provided with a plurality of holes.
